(19) United States
(12) Patent Application Publication
(10) Pub. No.: US 2026/0232100 A1
(43) Pub. Date: Aug. 13, 2026

(54) Support for Violin or Viola

(71) Applicant: Jeremiah Rose, Fitzroy North (AU)

(72) Inventor: Jeremiah Rose, Fitzroy North (AU)

(21) Appl. No.: 19/533,349

(22) Filed: Feb. 9, 2026

(30) Foreign Application Priority Data
Feb. 11, 2025 (AU) 2025900367

(51) Int. Cl. A45F 3/04; A61F 5/02

(52) U.S. Cl. CPC A45F 3/04; A61F 5/028

(57) ABSTRACT

A support for a violin or viola is provided. The support comprises a first strap having a first end configured to be fixedly attached, optionally releasably, to a lower-body clothing attachment point of a player and a second end configured to be fixedly attached, optionally releasably, to the instrument, the first strap being configured to pass around the player's back and over the shoulder region. In use, tension in the first strap applies to the instrument a force directed downwardly and toward the lower-body clothing attachment point, at or adjacent a button end of the instrument, to produce a levering action that urges a scroll end upward to support the instrument in a playing position. The support further comprises a second strap extending from the lower-body clothing attachment point to the instrument and configured to pass around a front of the player to resist rearward displacement of the instrument.

### CROSS-REFERENCE TO RELATED APPLICATIONS

This application claims priority to Australian Patent Application No. 2025900367, filed 11 Feb. 2025, the entire contents of which are incorporated herein by reference.

### TECHNICAL FIELD

The present invention relates to a support for a stringed instrument used by a musician intending to play that instrument whereby the support safely and securely holds the instrument in the usual playing position for a violin or viola, but without requiring the use of the chin or noting hand to hold the instrument erect.

### BACKGROUND OF THE INVENTION

Numerous supports are shown in the prior art for assisting a musician in holding a stringed instrument, particularly instruments in the violin family. Such supports generally seek to reduce the need for the player to use their chin and/or noting hand to hold the instrument in a playing position, thereby providing increased freedom of movement of the neck and noting hand. Such supports may be useful for players who experience discomfort when playing. However, many prior art supports exhibit one or more limitations that can reduce stability, adjustability or ease of use.

One such limitation arises where a support allows the instrument to rotate about the player's body or neck such that the instrument can move out of the playing position. For example, in WO2002035192A1 and U.S. Pat. No. 7,544 871B2, a strap passes around the body or neck of the player and may permit relative movement of the strap and instrument on the player.

Another limitation arises where the instrument is not adequately restrained against rearward displacement, which may allow the instrument to move rearwardly relative to the player. For example, U.S. Pat. No. 9,812,102B2 describes a support including strap elements associated with the player's torso and a connector to the instrument; however, rearward restraint of the instrument may be insufficient in some configurations.

Another limitation arises where a support is not easily and precisely adjustable to suit a player's individual technique. For example, in some prior art arrangements, an adjustment element is positioned across the upper back, making adjustment difficult while the support is in use.

Another limitation arises where movement of the player, including movement of the bowing arm, can result in undesirable movement of the instrument.

Another limitation arises where tension in a first strap acts in an unsuitable direction for achieving a desired playing position. In some prior art arrangements, strap tension may include a substantial horizontal component that tends to pull the instrument laterally, resulting in an unsuitable playing position.

Another limitation arises where the support is not compact enough to be stowed in a standard instrument case.

Another limitation arises where the support takes excessive time to fit or remove, delaying the player from beginning to play when required.

It is therefore desirable to provide a support that achieves an improved combination of features, including stable retention of the instrument in a suitable playing position, resistance to undesired rotation or rearward displacement, and convenient adjustability and compactness, thereby addressing limitations that may remain when such features are provided only individually or in incomplete combinations.

### SUMMARY OF THE INVENTION

In one aspect, the invention provides a support for a violin or viola comprising a first strap comprising one or more strap portions, having a first end configured to be fixedly attached, optionally releasably, to the player's lower-body clothing and a second end configured to be fixedly attached, optionally releasably, to the instrument, the first strap further including a tensioning mechanism for adjusting tension between said first and second ends. The first strap is configured, in use, to pass around the player's back and over the shoulder region. The support further comprises a second strap having a first end configured to be fixedly attached, optionally releasably, to the same lower-body clothing attachment point as the first end of the first strap, and a second end configured to be fixedly attached, optionally releasably, to the instrument, the second strap being configured, in use, to pass around a front of the player.

The first strap is configured to secure the instrument in a stable playing position through the action of a force directed downwardly and toward the lower-body clothing attachment point arising from the tension between said first and second ends. Said force results in a levering action: as the bottom end (button end) of the instrument is pulled downwards toward the lower-body clothing attachment point by the first strap, the top end (scroll end) is levered upwards, with the fulcrum of said levering action being located between the two ends, either through the customary use of a shoulder rest, or by some other means. This configuration of the first strap effectively holds the instrument in the playing position and prevents the instrument from sliding forwards around the player's body.

The tensioning mechanism on the first strap is located so that it is reachable with the player's bowing hand while the support is in use. This allows the instrument to be raised or lowered according to the player's preference, so that adjustments can be made during use without needing to remove the support.

The first strap and second strap may optionally be implemented as portions of a single continuous strap that is configured, in use, to pass around the player's back, over the shoulder region and around the front of the player. In these configurations, the single continuous strap is configured to be fixedly attached, optionally releasably, to the player's lower-body clothing and to the instrument.

The attachment between the first strap and the second strap and the player's lower-body clothing may be achieved by means of a clip configured to engage a belt loop of the player, thereby providing a common lower-body clothing attachment point for the first strap and the second strap, or by other attachment means. In some embodiments, the clip or other attachment means is alternatively configured to engage a separate strap or loop worn on the player's lower body, including a leg loop.

### BRIEF DESCRIPTION OF THE DRAWINGS

FIG. 1 illustrates a preferred embodiment of the support operatively attached to a violin and to the player's belt loop, viewed from the front.

FIG. 2 is a more detailed view of FIG. 1, showing a preferred embodiment of the support being attached to the player's belt loop, viewed from the side.

FIG. 3 illustrates a preferred embodiment of the support operatively attached to a violin and passing around the player's back and shoulder, viewed from the back.

FIG. 4 illustrates a preferred embodiment of the support operatively attached to a violin by means of a removable clamp and clip.

FIG. 5 shows an alternative embodiment of the support being attached to the player's lower body by means of an additional strap configured to loop around a portion of the player's leg.

### DETAILED DESCRIPTION OF EMBODIMENTS

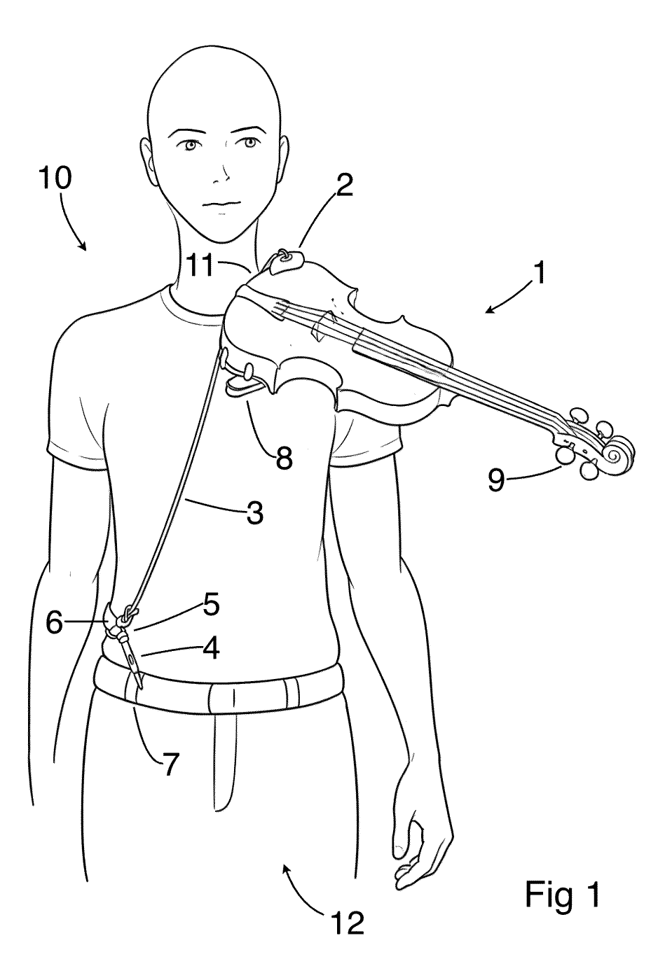

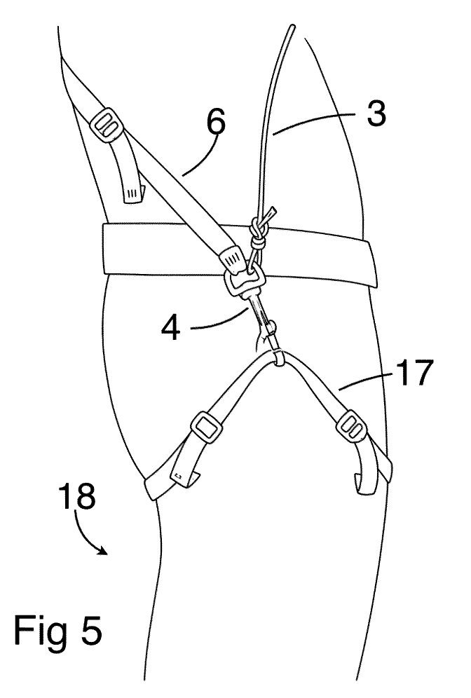

A preferred embodiment of the support of this invention is generally indicated in FIGS. 1-4, and an alternative embodiment in FIG. 5.

In the view of FIG. 1, a preferred embodiment of the second strap 3 is shown operatively passing around the front of the player 10, with a violin 1 shown in the playing position. In the illustrated embodiment, the second strap 3 comprises a flexible cord. In other embodiments, the second strap 3 may comprise webbing, string, rope or similar flexible material, and may include an adjustment mechanism. In use, the second strap 3 is configured to resist rearward displacement of the violin 1 relative to the player 10.

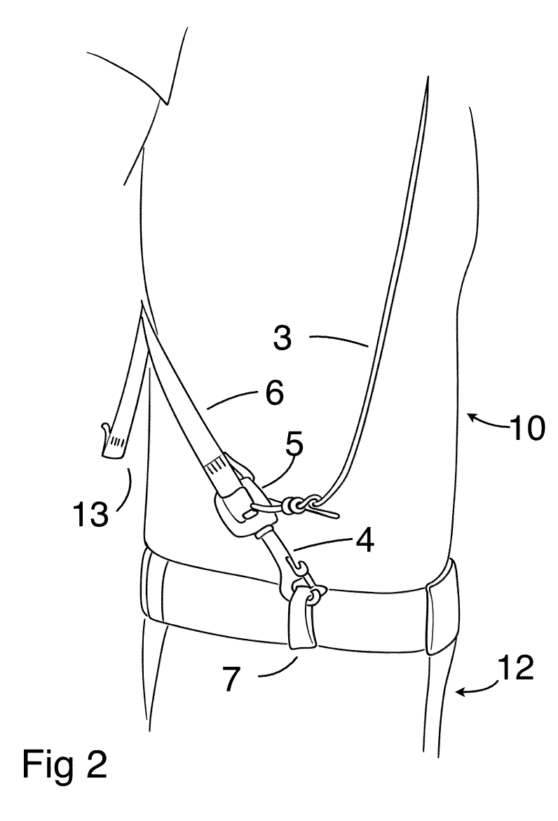

The view of FIG. 2 shows the support being fixedly attached, optionally releasably, to a belt loop 7 of the player's pants 12. In the illustrated embodiment, the support is removable by use of a clip 4 having a ring 5, wherein both the first strap 6 and the second strap 3 are attached to the ring 5, thereby providing a common lower-body clothing attachment point.

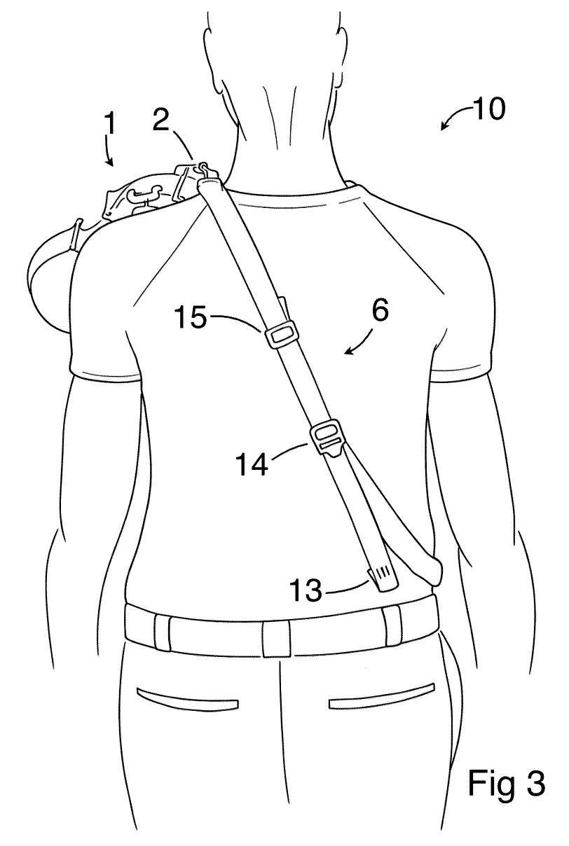

In the view of FIG. 3, a preferred embodiment of the first strap 6 is shown passing around the back of the player 10 and over the shoulder region, and attached to the violin 1 by means of a removable clamp 2. In the illustrated embodiment, the first strap 6 comprises nylon webbing; in other embodiments, the first strap 6 may comprise string, cord, rope or similar flexible material. The first strap is tensioned by a buckle mechanism, for example a ladderlock 14, such that the user can tighten the support by pulling an adjustment strap 13 and can loosen the support by lifting the ladderlock 14. The tensioning mechanism 14 is preferably positioned so that it can be reached by the player while the support is in use. In the illustrated embodiment, a slide 15 is used to adjust the overall length of the first strap as an additional adjustment mechanism to the tensioning mechanism.

In use, tension in the first strap 6 applies to the violin 1 a force directed downwardly and toward the lower-body clothing attachment point 7, at or adjacent a button end 11 of the violin, to produce a levering action that urges a scroll end 9 upward, with a shoulder rest 8 or contact at the player's shoulder region acting as a fulcrum.

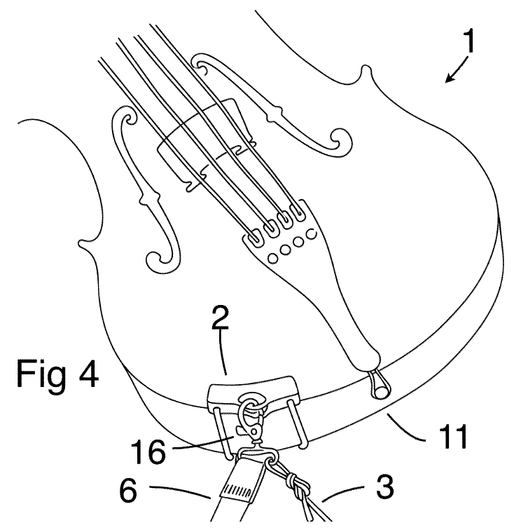

The view of FIG. 4 shows one possible embodiment of the first strap 6 and second strap 3 being fixedly attached, optionally releasably, to a violin 1 by a clip 16 connected to a clamping mechanism 2 on the violin 1. Other attachment methods may be used, including a loop of cord engaged with a hook fixed or clamped to the instrument, or another coupling arrangement, provided the attachment enables the levering action described above by applying the downwardly directed force at or adjacent the button end.

In an alternative embodiment (for example FIG. 5), the first strap 6 and second strap 3 are fixedly attached, optionally releasably, to an additional leg-loop strap 17 configured to pass around a portion of the player's leg 18. Such an embodiment may be advantageous where the player does not have suitable belt loops or other attachment features on the player's lower-body clothing.

Wherever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this invention have been described, it will be evident to those skilled in the art that the present invention may be embodied in other specific forms without departing from the essential characteristics thereof. The present embodiments and examples are therefore to be considered in all respects as illustrative and not restrictive, and all modifications which would be obvious to those skilled in the art are therefore intended to be embraced therein.

1. A support for a violin or viola, comprising:
a first strap having a first end configured to be fixedly attached, optionally releasably, to a lower-body clothing attachment point of a player and a second end configured to be fixedly attached, optionally releasably, to the violin or viola, the first strap being configured, in use, to pass around the player's back and over the shoulder region of the player; and
a second strap having a first end configured to be fixedly attached, optionally releasably, to the same lower-body clothing attachment point and a second end configured to be fixedly attached, optionally releasably, to the violin or viola, the second strap being configured, in use, to pass around a front of the player;
wherein the first strap includes a tensioning mechanism for adjusting tension in the first strap; and
wherein, in use, tension in the first strap applies to the violin or viola a force directed downwardly and toward the lower-body clothing attachment point at or adjacent a button end of the violin or viola to produce a levering action that urges a scroll end of the violin or viola upward about a fulcrum located between the button end and the scroll end at or adjacent the shoulder region of the player, thereby supporting the violin or viola in a playing position.

2. The support of claim 1, wherein the lower-body clothing attachment point comprises a belt loop of the player.

3. The support of claim 1, wherein the first end of the first strap and the first end of the second strap are attached to a common attachment member configured to engage the lower-body clothing attachment point.

4. The support of claim 2, wherein the first end of the first strap and the first end of the second strap are attached to a common attachment member configured to engage the lower-body clothing attachment point.

5. The support of claim 3, wherein the common attachment member comprises a clip having a ring.

6. The support of claim 4, wherein the common attachment member comprises a clip having a ring.

7. The support of claim 1, wherein the first strap comprises one or more strap portions forming a tensile load path between the first end and the second end of the first strap.

8. The support of claim 1, wherein the tensioning mechanism comprises a buckle mechanism.

9. The support of claim 8, wherein the buckle mechanism comprises a ladderlock.

10. The support of claim 1, wherein the tensioning mechanism is positioned so that it is reachable by the player with the bowing hand while the support is in use.

11. The support of claim 1, wherein the second strap includes an adjustment mechanism for adjusting an effective length of the second strap.

12. The support of claim 1, wherein at least one of the first strap and the second strap comprises webbing.

13. The support of claim 1, wherein at least one of the first strap and the second strap comprises cord, string or rope.

14. The support of claim 1, wherein the second end of the first strap and the second end of the second strap are configured to be attached to the violin or viola via a removably secured attachment device.

15. The support of claim 1, wherein the second end of the first strap and the second end of the second strap are configured to be attached to a chin rest assembly of the violin or viola.

16. The support of claim 1, wherein the lower-body clothing attachment point is alternatively provided by a separate strap or loop worn on the player's lower body, including a leg loop, to which the first end of the first strap and the first end of the second strap are attachable.

17. The support of claim 1, wherein the second strap is configured, in use, to resist rearward displacement of the violin or viola from the playing position.

18. A method of supporting a violin or viola, comprising:
fixedly attaching, optionally releasably, a first end of a first strap to a lower-body clothing attachment point of a player;
fixedly attaching, optionally releasably, a second end of the first strap to the violin or viola;
routing the first strap around the player's back and over the shoulder region of the player;
tensioning the first strap such that a force is applied to the violin or viola directed downwardly and toward the lower-body clothing attachment point at or adjacent a button end of the violin or viola to produce a levering action that urges a scroll end upward about a fulcrum at or adjacent the shoulder region of the player;
fixedly attaching, optionally releasably, a first end of a second strap to the lower-body clothing attachment point;
fixedly attaching, optionally releasably, a second end of the second strap to the violin or viola; and
routing the second strap around a front of the player.
